(19) United States
(12) Patent Application Publication
(10) Pub. No.: US 2017/0205352 A1
(43) Pub. Date: Jul. 20, 2017

(54) DETECTION OF EXPLOSIVES USING RAMAN SPECTROSCOPY WITH GOLD/SILVER NANOSPONGE ALLOY

(71) Applicant: OCEAN OPTICS, INC., Dunedin, FL (US)

(72) Inventors: DAVID CREASEY, SAFETY HARBOR, FL (US); DEREK GUENTHER, ST. PETE BEACH, FL (US)

(21) Appl. No.: 15/072,450

(22) Filed: Mar. 17, 2016

Related U.S. Application Data
(60) Provisional application No. 62/137,340, filed on Mar. 24, 2015.

(51) Int. Cl. G01N 21/65; G01N 33/22

(52) U.S. Cl. CPC G01N 21/65; G01N 33/227

(57) ABSTRACT

A system for detection of low concentration of explosives via Raman spectroscopy is disclosed that is more specifically a Raman detection substrate having a surface-enhancement effect by using roughened glass (or “frosted” glass) with a gold/silver nanosponge alloy sputter-deposited onto it under increased pressure resulting in a nano-porous structure to hold the analyte.

### CROSS-REFERENCE TO RELATED APPLICATION

The present application claims the benefit of previously filed co-pending Provisional Patent Application Ser. No. 62/137,340, filed on Mar. 24, 2015.

### FIELD OF THE INVENTION

This invention belongs to the field of detection of low concentration of explosives via Raman spectroscopy. More specifically it is a detection substrate design having a surface-enhancement effect by using roughened glass (or “frosted” glass) with a gold/silver nanosponge alloy sputter-deposited onto it.

### BACKGROUND OF THE INVENTION

Sputtering is a method of thin film deposition that utilizes a high-vacuum plasma phase to slowly and steadily eject a material (in this case gold/silver) from a bulk target onto a substrate opposite that target. Many different working gases and targets can be used. Gold and silver are known to have Raman surface enhancing effects with various compounds, and one can co-sputter both of these metals at the same time. Silver is of course far less expensive than gold, so a silver target with gold foil strips overlaid over a portion of the area is used.

The following discloses the use of a Gold/Silver nanosponge alloy which, when sputtered onto a frosted glass substrate and incorporated into the design of a Raman detection system, greatly facilitates detection of explosives. An appreciation of the advantages these features represent when compared with previous designs can be derived from consideration of the following drawings and description of the invention.

### BRIEF SUMMARY OF THE INVENTION

This invention is a Raman spectrometer design having a surface-enhancement effect by using roughened glass (or “frosted” glass) with a gold/silver nanosponge alloy sputter-deposited onto it.

### BRIEF DESCRIPTION OF THE DRAWINGS

For a fuller understanding of the nature and objects of the invention, reference should be made to the following detailed description, taken in connection with the accompanying drawings, in which:

FIG. 1 shows a view of the sputtering system of the preferred embodiment; and,

FIG. 2 shows a view of the sputtering target of the preferred embodiment.

### DESCRIPTION OF THE PREFFERED EMBODIMENT

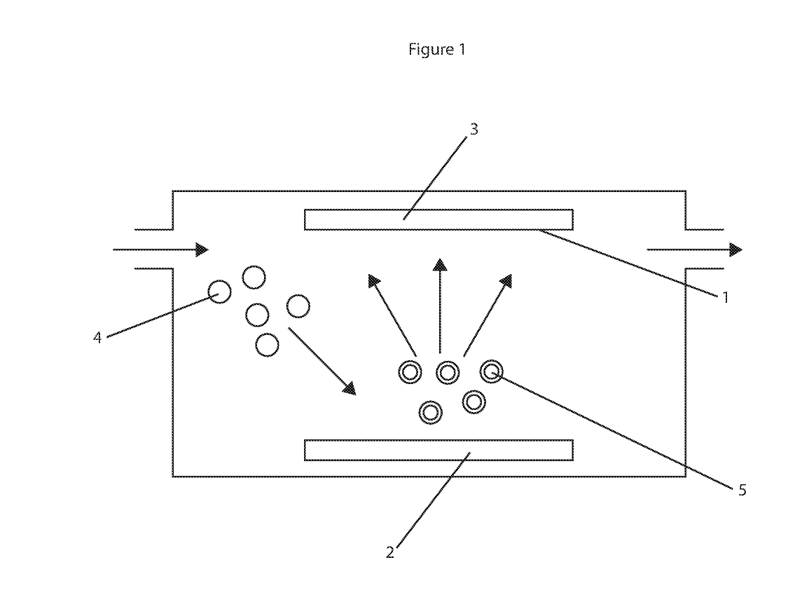

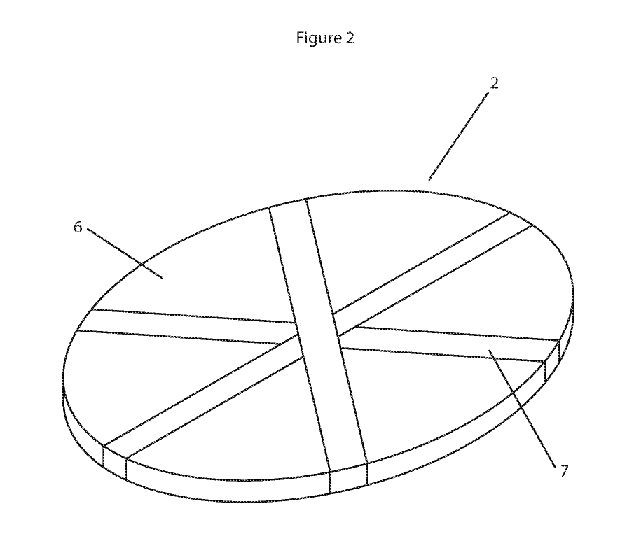

What is disclosed herein is the use of a thin film Gold/Silver nanosponge (1) for improving Raman spectroscopy performance in explosive detection and, as disclosed in the preferred embodiment, is a Raman detection substrate design that uses Argon gas (4) sputtering (as shown in FIG. 1) of Gold/Silver sputtering target (2) (as shown in FIG. 2) onto a frosted glass substrate (3) to form a Gold/Silver nanosponge (1) to hold an analyte.

Sputtering is a method of thin film deposition that utilizes a high-vacuum plasma phase to slowly and steadily eject a material (in this case gold/silver (5)) from a bulk target (2) onto a frosted glass substrate (3) opposite that target (2). Many different working gases and targets can be used, but the preferred embodiment uses an argon plasma environment of around 6 mTorr.

Gold (7) and silver (6) are known to have Raman surface enhancing effects with various compounds, and one can co-sputter both of these metals at the same time. Silver (6) is far less expensive than gold (7), so the preferred embodiment target (2) uses a silver disc (6) with gold foil strips (7) overlaid over a portion of the area as shown in FIG. 2.

Sputtering is truly an art in that there are many parameters that can be tweaked to achieve all types of coatings and structures. One of these parameters that has been exploited in the past for a completely different application is the working gas pressure. As the amount of argon in the chamber increases, the bombarded gold/silver atoms ricochet off of these argon atoms to create a looser “sponge-like” structure; conversely as the pressure approaches perfect vacuum the coating becomes much denser. Thus working at a higher-than-usual pressure creates a nano-porous structure for the analyte to sit within. Optimizing this working gas pressure to determine ideal porosity can easily be accomplished by one skilled in the art.

After about 15 minutes of sputtering in a chamber a Gold/Silver nanosponge (1) film of roughly 150 nm can be deposited onto the frosted glass substrate (3) surface. This film has a very attractive gold/silver-fusion color to it, which is expected from the co-deposition.

Sputtered substrates have a number of key advantages over some of the prior art paper-based technology, including:
Ease of fabrication: No chemicals, no wet chemistry, no multi-step synthesis;
Quickness of fabrication: 15 minutes can generate about 5000 mm² (in current chamber) of consistent coating, or 200 samples at 5 mm×5 mm active area;
Cost of fabrication: One can get very good results at the initial thickness of 150 nm, which is a negligible amount of material whether gold or silver ends up being the more attractive metal;
Repeatability of fabrication: The simplicity of the approach, quite literally a single step, cuts out all of the variables found in the prior art multi-step nano-particle ink approach;
Immediate use: The ink-based approach has been found to need about 45 days of curing before optimal performance; in the case of the sputtered samples as soon as they come out of the chamber they are ready to use.

A 785 nm Raman system is used along with the preferred embodiment detection substrate for detection of explosives, though the presence of silver suggests that the 532 nm system is also worthwhile. In testing of the preferred embodiment detection substrate a QE Pro bench was set to a 5 second integration time, and the arbitrary power setting on the laser was set to 0.40. The blank detection substrate was placed underneath the focused laser probe and the “dark” reference was taken with the laser on to eliminate any background signal. A small volume (10 μL) of sample explosive solution in solvent was then dropped onto the surface where the laser dot was focused. It should be noted that care should be taken not to physically move the substrate or laser as it is key that once the “dark” is taken with the laser on all that happens to the system is the un-shifted addition of sample liquid.

A solvent such as acetone evaporates very quickly so the user can expect to see initial jumps in signal when the solvent is first present, but after several full 5-second scans this retreats back down and leaves the analytical peaks behind.

The most promising explosive tested thus far is RDX. While there are variations in analytical peak intensity across the sputtering runs, and even the roughness across a single substrate, one can consistently see RDX activity on all samples tested and it is a consistently repeatable effect.

The limit of detection was briefly investigated with the small amount of RDX solution and testable substrates available, and it was shown that signature peaks are detectable down to 5*10⁻⁷ M RDX, though not detectable at 1*10⁻⁷ M.

Similar tests on PETN also show two signature peaks that were consistently present in the majority of samples. While they are not far above the background noise, they are almost always the two maxima in the observed spectral range.

There are a massive number of parameters than can be optimized for this explosive detection platform as can easily be determined by those skilled in the art. Areas of optimization include:
Alloy distribution: Weight of gold versus silver;
Alloy distribution per Raman system: Weight of gold versus silver at 785 nm and 532 nm;
   (Palladium nanosponge performance at 532 nm);
Film thickness: Variably timed deposition runs;
Film porosity: Variable working gas pressures;
Glass roughness: Study over wide range of roughness standards;
Roughness per material: Study over selected range of roughness across wider range of materials, including glass, quartz, silicon, and polymers;
   If a roughened polymer with gold/silver deposition can achieve the same effect, cost of material and processing (i.e. cutting) is immediately reduced by a drastic amount.

Since certain changes may be made in the above described Raman detection substrate design for explosive detection without departing from the scope of the invention herein involved, it is intended that all matter contained in the description thereof or shown in the accompanying figures shall be interpreted as illustrative and not in a limiting sense.

What is claimed is:

1. A detection substrate device used with Raman spectroscopy to detect explosives comprising:
a roughened substrate; and,
said roughened substrate coated with a nano-porous thin film of gold and silver alloy.

2. The detection substrate of claim 1 wherein said roughened substrate is a roughened glass substrate.

3. A method of making a detection substrate device used with Raman spectroscopy to detect explosives comprising:
roughening a substrate; and,
then sputtering a nano-porous thin film of gold and silver alloy on said roughened substrate in a high-vacuum phase argon plasma environment.

4. The method of making the detection substrate of claim 3 wherein said roughened substrate is a roughened glass substrate.

5. A method of detecting explosives comprising;
placing a roughened glass substrate coated with a thin film of gold and silver alloy underneath a focused laser probe of a Raman spectroscope and recording reference analytical peak intensities;
placing a test sample on said roughened glass substrate underneath a focused laser probe of a Raman spectroscope and recording sample analytical peak intensities; and, then comparing said reference analytical peak intensities to said sample analytical peak intensities to determine the presence of explosives.
